(19) United States
(12) Patent Application Publication
(10) Pub. No.: US 2018/0100137 A1
(43) Pub. Date: Apr. 12, 2018

(54) AUTOMATED CULTURE MEDIA PREPARATION SYSTEM AND METHOD FOR MICROBIOLOGY TESTING

(71) Applicant: Heateflex Corporation, Arcadia, CA (US)

(72) Inventors: Hector Joel Castaneda, Cypress, CA (US); Jorge Ramirez, Glendale, CA (US)

(73) Assignee: Heateflex Corporation, Arcadia, CA (US)

(21) Appl. No.: 15/287,972

(22) Filed: Oct. 7, 2016

(51) Int. Cl. C12N 1/20; C02F 1/00; C02F 1/02; C02F 1/32; B01D 36/00

(52) U.S. Cl. CPC C12N 1/20; C02F 1/001; C02F 1/02; C02F 1/008; C02F 2303/04; B01D 36/001; C02F 2209/006; C02F 2209/02; C02F 1/32

(57) ABSTRACT

A process and system for automated culture media preparation for microbiology testing includes providing a non-pressurized water tank having an air filter associated with the air vent thereof. Bacteria within the water is filtered, killed and/or disabled. The water is heated to a predetermined temperature. A predetermined amount of the purified and heated water is dispensed into a container for mixing with a culture media. The water dispensed into the container is weighed. Information relating to the temperature, volume and weight of the dispensed water and the culture media to be mixed with the dispensed water is electronically stored. A label having indicia, including the electronically stored information, is printed.

### BACKGROUND OF THE INVENTION

The present invention generally relates to microbiology testing, such as food microbiology testing using culture or growth media. More particularly, the present invention is related to an automated culture media preparation system and method for microbiology testing.

Food contaminated with certain organisms can cause discomfort, illness and even death to the consumer. To ensure safety, food processors and food testing laboratories conduct microbiology testing to verify the absence of common pathogenic microbes, such as E. coli, listeria, salmonella and the like. Food processors can either perform the testing in-house or outsource the task to testing laboratories. In both cases, the present state of testing for pathogens has been a slow, manual process with risk of human error.

Traditionally, testing is done on a food sample. The sample is collected, weighed and placed in a sterile container, such as a beaker or bag. The sample is labeled and manually logged or recorded. Next, media is weighed then added to the container with the food sample. The media is often referred to as culture media, growth media or enrichment media that has specific nutrients that support the growth of one or more specific organisms, at times to the exclusion of other microbes. Detection of a target organism can occur by selecting the correct enrichment media and adding it to a food sample. If present, the target microorganism on the food sample will become more apparent as growth is encouraged by the media.

It is desirable to add the media in the correct proportion to the volume of the food sample. In the past, the amount of enrichment or culture media has been manually recorded by weight or volume, along with the media batch identifier. Variations or inaccurate mixtures may provide false or incorrect test results.

In some systems and methods, the media is prepared as a batch by mixing proportions of dry media and water. This requires that the human operators provide the correct proportions, and there is nothing to verify that the proportions were added exactly and correctly. The batch system works for one testing recipe. Once the batch is used up, another batch must be prepared. However, as media has a shelf life, if different testing recipes for different pathogens are required, this requires creating multiple batches of liquid media. Large food samples can require multiple liters of media, and thus the prepared batch may be used up relatively quickly, or in some cases multiple batches are required to be prepared.

In other cases, a dry media is added and a precise volume of ultra-pure water is added to the food sample and the culture media within the container. Dry media is often preferred as it has a longer shelf life, is much less expensive than liquid or previously prepared media, and can be obtained in packets of a particular volume or weight. Adding the precise volume of ultra-pure water is done by the operator visually measuring the water volume, such as with graduated cylinders filled by a peristaltic pump.

Finally, the food sample and the culture or enhancement media is incubated, isolated and analyzed for specific bacteria counts, documented and later the waste is autoclaved.

The currently used systems and methods of food microbiology testing provide many drawbacks. In the batch preparation systems, only a portion of the batch may be used while the remainder exceeds its shelf life and must be discarded. The batch preparation devices must be cleaned thoroughly between the preparation of each batch. Moreover, in both the batch preparation systems as well as the dry enrichment media methodologies, there is a risk of human error in correctly adding the exact proportions of media and water or during the manual process of logging the information relating to the food microbiology test being performed.

As government and industry regulations change and demand increases for more food testing, improvements need to be made to safely increase the throughput of food testing by automating some of the processes and the required documentation. There is also a need for a system and methodology that reduces human errors, increases, throughput and thereby profits, and improves the traceability for sample preparation and documentation. The present invention fulfills these needs, and provides other related advantages.

### SUMMARY OF THE INVENTION

The present invention relates to an automated culture media preparation system and methodology for microbiology testing, such as food microbiology testing. The present invention prepares laboratory-grade water and dispenses it at the proper volume and temperature to obtain consistently accurate testing results while automatically recording the identity of the user, sample, media and recording the actual process parameters used in conjunction with the testing for later verification, if needed.

The automated culture media preparation system for microbiology testing of the present invention generally comprises a water tank, including a water inlet, a water outlet and an air vent, which filters the air between the atmosphere and the non-pressurized water tank. An air filter is associated with the air vent for filtering bacteria from the air entering the water tank. Preferably, the air filter associated with the air vent comprises an ultra-low penetration filter capable of filtering objects having a size greater than 0.2 microns.

A water dispenser is in fluid communication with the water tank. A pump circulates and moves the water from the water tank to the dispenser. Means are provided for filtering, killing and/or disabling bacteria within the water. Such means may comprise a filter capable of filtering bacteria from the water and/or a source of energy that kills or disables the bacteria. A source of ultraviolet energy that kills or disables the bacteria may be used. These may be disposed in series between a source of water upstream of the water tank inlet and the water dispenser. Multiple filters may be used to filter objects of different sizes from the water. For example, a first filter may filter the water of objects having a size of greater than one micron, and a second filter filters the water of objects having a size greater than 0.2 microns.

The system also includes an electronic controller. The electronic controller may be in electronic communication with a heater for heating the water to a selected temperature. A water temperature sensor may also be operably coupled to the electronic controller. The electronic controller may comprise a programmable logic controller having memory associated therewith.

Means are provided for controllably dispensing a selected amount of water from the water dispenser. Such means may comprise a flow sensor and a valve operably coupled to the electronic controller.

A scale is associated with the water dispenser, and communicates with the electronic controller so as to record the weight of the water dispensed into a container placed on the scale in memory associated with the electronic controller.

A data entry device, such as a keypad, a touch screen, and/or a machine code reading scanner, is in electronic communication with the electronic controller. A printer for printing labels is also in electronic communication with the electronic controller.

The process for preparing media for microbiology testing in accordance with the present invention comprises the steps of providing a source of water. This may comprise the step of providing a water tank exposed to ambient air and filtering the air entering the water tank for objects at least the size of a bacteria.

The water is purified of pathogenic bacteria. This may comprise the steps of filtering, disabling, and/or killing the bacteria within the water. This may be done by passing the water through a filter that filters bacteria from the water. The water may be passed through a series of filters so that objects that are at least 0.2 microns in size are filtered from the water. The purifying step may additionally, or alternatively, comprise the step of exposing the water to ultraviolet light to kill or disable bacteria within the water.

The water is heated to a predetermined temperature. For example, the water may be heated to a temperature corresponding with an incubation temperature of bacteria to be tested for.

To prevent water stagnation, the water may be circulated through a device that stores and dispenses the water. The device that stores and dispenses the water may be sterilized by heating the water to a temperature sufficient to kill or disable bacteria and circulating the heated water through the device.

A predetermined amount of the purified and heated water is dispensed into a container for mixing with a culture media. The water that is dispensed into the container is weighed.

Information relating to the temperature, volume and weight of the dispensed water and the culture media to be mixed with the dispensed water is electronically stored. A label having indicia, including the electronically stored information, is printed.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a front perspective view of an automated media preparation system embodying the present invention;

FIG. 2 is a front elevational view of the system, with a front panel removed to expose internal components thereof;

FIG. 3 is a side elevational view of the system embodying the present invention, illustrating various components thereof;

FIG. 4 is an opposite side view of FIG. 3, illustrating various components thereof;

FIG. 5 is a schematic plumbing diagram of the system of the present invention;

FIGS. 6A through 6F are schematic diagrams of an electronic controller in electronic communication with various components of the present invention;

FIGS. 7A through 7G are additional electronic schematics illustrating an electronic controller in communication with various components of the present invention; and

FIG. 8 is an exemplary label printed in accordance with the present invention.

### DETAILED DESCRIPTION OF THE PREFERRED EMBODIMENTS

The present invention resides in an automated culture media preparation system, and method, for microbiology testing. The system of the present invention provides testing laboratories with automation and traceability in media preparation used for microbiology testing, such as food microbiology testing. The present invention is used in the field of microbe pathogen testing by preparing laboratory-grade water which is dispensed at the proper volume and temperature to obtain consistently accurate testing results, while automatically recording actual process parameters, including the identity of the user, sample, and media, water volume dispensed, and temperature of the water.

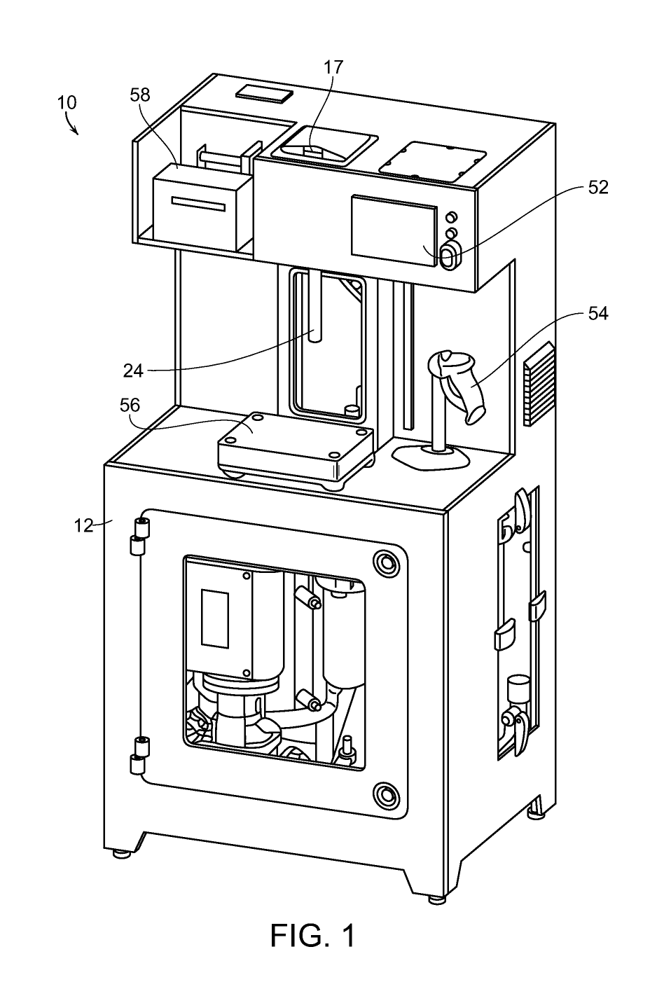

With reference now to FIGS. 1-4, an exemplary system embodying the present invention is shown. The system is typically in the form of a stand-alone unit 10 having many of the components thereof housed within an enclosure or housing 12.

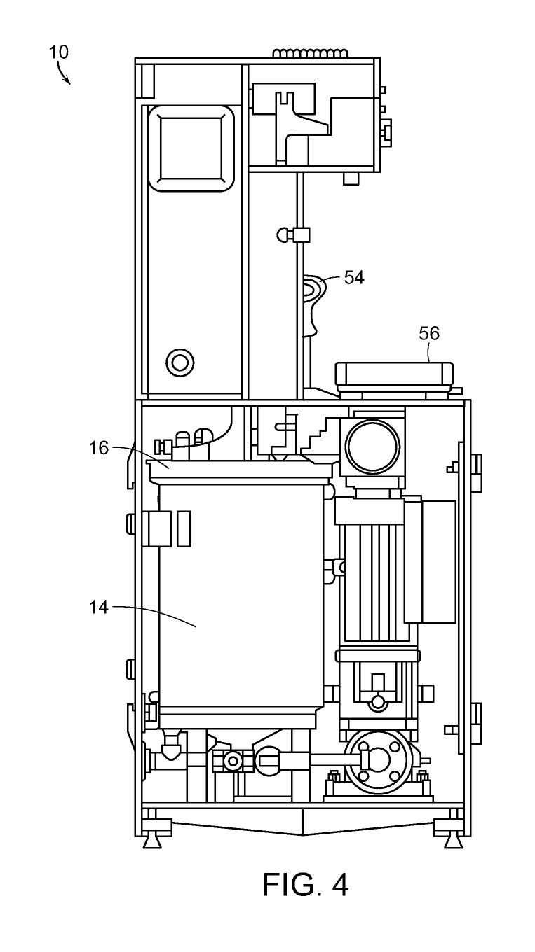

As illustrated in FIG. 4, the system includes a water tank 14. In a particularly preferred embodiment, the water tank is a non-pressurized water tank, which is exposed to filtered ambient air so that the ambient air can be drawn into the water tank 14 as water is removed therefrom. Air is expelled from the water tank 14 as water fills the tank 14. In order to avoid contamination, an air filter 16 is associated with an air vent 17 of the water tank 14 so as to filter bacteria and other objects from the air, preventing their entry into the water tank 14. In a particularly preferred embodiment, a high purity filter, such as an ultra-low penetration filter is used which is capable of filtering objects having a size greater than 0.2 microns. The ultralow penetration air filter (ULPA) 16 can remove from the air at least 99.999% of dust, pollen, mold, bacteria and any airborne particles with a size of 0.1 microns or larger. The filter reduces or eliminates the spread of airborne pathogens into the water in the system. This arrangement overcomes the drawbacks and difficulties used in association with a closed, pressurized tank, while providing the benefits of an open-air tank that would otherwise potentially be contaminated.

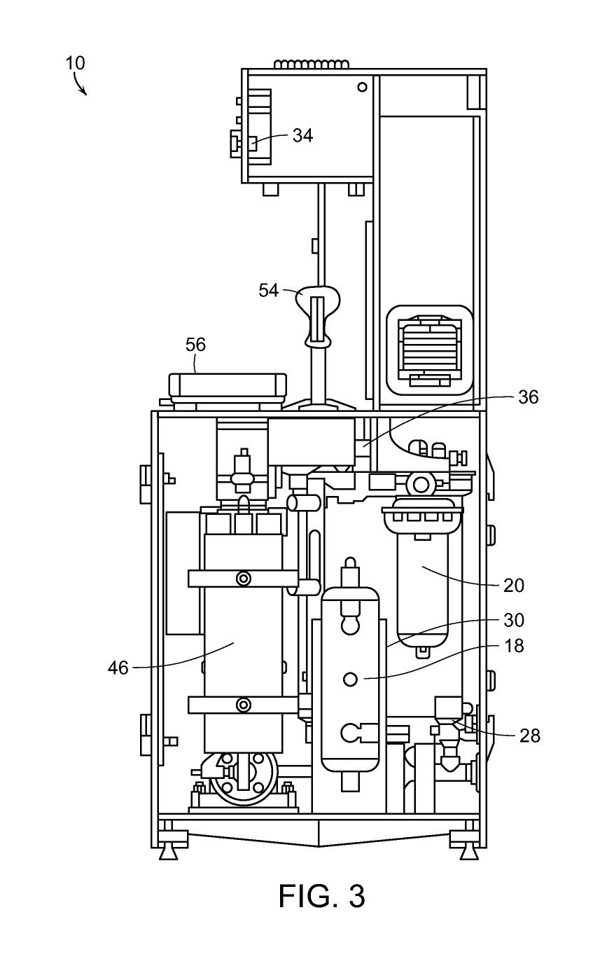

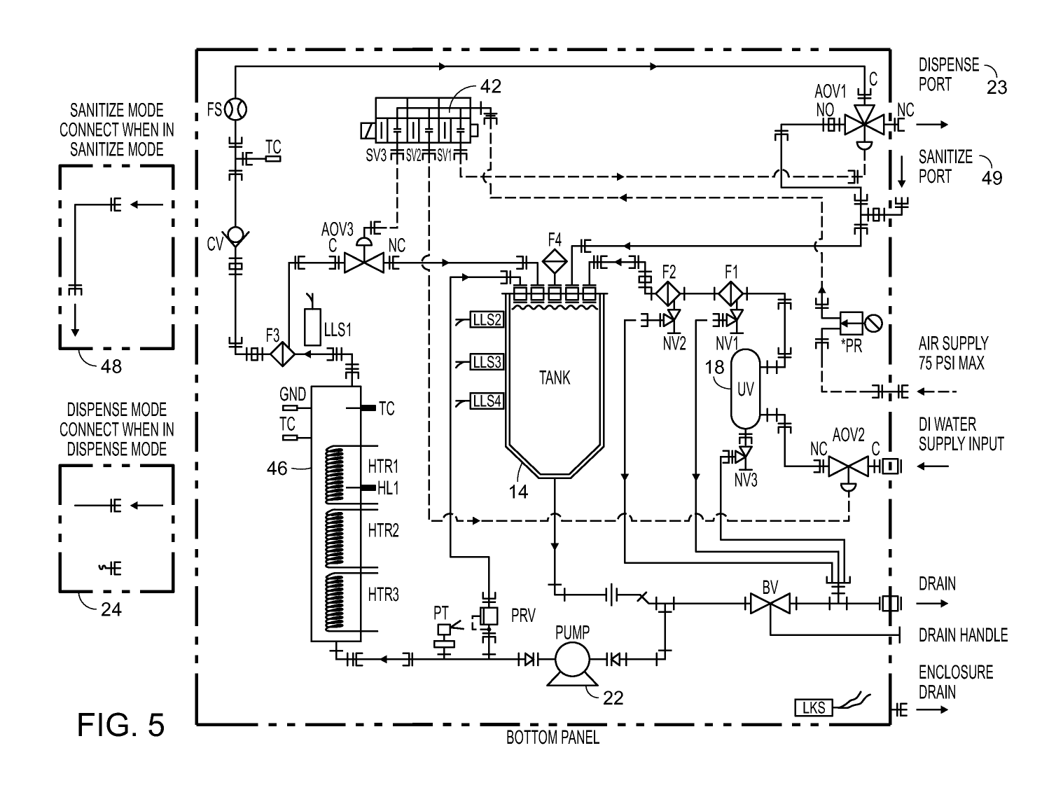

With reference now to FIG. 3, preferably the water is purified before entering the inlet of the water tank 14. This may be done by filtering the water and/or exposing the water to a source of energy that would kill or disable bacteria within the water. In a particularly preferred embodiment, an ultraviolet light source, emitted from an ultraviolet light bulb emits ultraviolet light 18 through the water before it enters the water tank. Moreover, the water is passed through at least one filter of a filter cartridge 20. In a particularly preferred embodiment, the water passes through a series of filters (F1 and F2 in FIG. 5) before entering the water tank 14. Typically, a first filter (F1) will filter objects of a relatively larger size, such as 1.0 microns, whereas a second (F2) or final filter 21 (F3) will filter the water of smaller objects, such as 0.1 microns so that the water is purified, laboratory grade and clear of bacteria that could otherwise contaminate the sample and create errors in testing and readings. While a single filter cartridge 20 is illustrated and may include multiple filters and possibly multiple water pathways, it will be appreciated by those skilled in the art that the individual filters could have their own cartridges separate from one another.

A pump assembly 22 is used to circulate the water through the system, such as at least between the water tank 14 and an outlet dispenser 24. Typically, the pump assembly 22 will be used to pump water through the ultraviolet light assembly 18, the filter cartridge 20, into the water tank 14, and throughout the remaining plumbing of the system to the outlet port 23 and dispenser 24. The pump may be a multi-stage centrifugal type, having a variable drive frequency.

As illustrated in FIG. 5, which is a plumbing schematic of the system of the invention, numerous valves, regulators, solenoids, pressure transducers and the like are used to control the flow of the water through the system. For example, such valves may include a pressure relief valve 26, an air operated valve 28, a needle valve 30, valve diaphragms 32 and 34, valve chuck 36, and valve ball 38. Other components which control the flow of fluid through the system include a pressure regulator 40 and one or more solenoid valves 42, typically three solenoid valves being used.

In order to avoid stagnation, water is generally circulated throughout the system, on at least a periodic basis, even if water is not being dispensed through the outlet dispenser 24. The system also includes a drain 44 for draining the water tank 14 and system of all water if desired or necessary, such as during repair or maintenance of the unit 10.

In a particularly preferred embodiment, the water is heated before being dispensed. This may be done, for example, by heating the water within the water tank 14 and system to a level between the ambient room temperature and the incubation temperature of the microbe to be tested for, and then raising the temperature of the water to the higher desired level before dispensing. For example, E. coli O157:H7 is incubated at approximately 42° C. This is much higher than ambient room temperature, which is typically approximately 24° C. Thus, the water within the tank 14 and system may be heated to a higher temperature than ambient room temperature before dispensing. The water may be heated to the incubation temperature, in the case of the E. coli strain approximately 42° C. so that it is dispensed at essentially the incubation temperature.

On the other hand, in some cases, preheating the water may not be necessary, as the incubation temperature is slightly above typical ambient room temperature, such as the case of Listeria, which has an incubation temperature of approximately 27° C. In this case, the water in the tank 14 could be maintained at ambient room temperature, and optionally slightly heated to approximately 27° C. before dispensing. Alternatively, the water in the water tank 14 and the entire system could be heated to approximately 27° C., or even dispensed at ambient room temperature which is typically only a few degrees lower than the incubation temperature of Listeria. The owner and operator will be able to select the temperature at which the water within the water tank 14 and system is maintained, and the microbe pathogen to be tested for will determine the final water temperature that is dispensed.

As such, the system includes a heater module 46 which serves to heat the water as explained above. The heater module may contain multiple heaters, for example, to heat the water at a maintained temperature and an elevated dispense temperature. Alternatively, the heater may only be used to heat the water to be dispensed to a predetermined temperature, which typically corresponds to the incubation temperature of the microbe pathogen to be tested form.

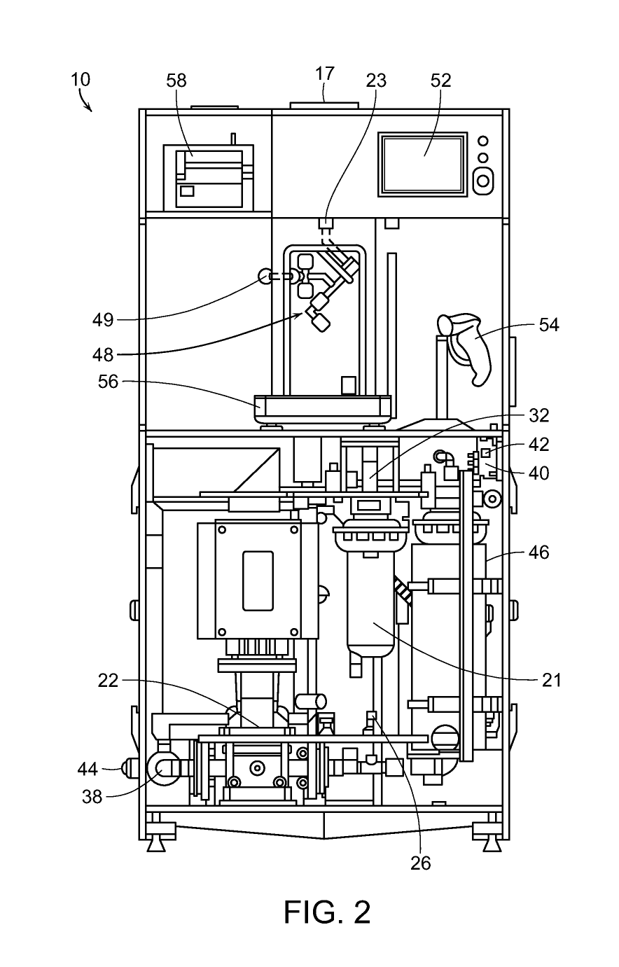

The entire system may be periodically sterilized by heating the water within the system to a temperature which will kill all microbes that would otherwise interfere with the microbiology testing. For example, the water can be heated to at least 75° C., and more preferably above 90° C., such as 94° C., so as to ensure that all bacteria which may potentially be present within the water tank 14 and throughout the entire system which comes into contact with the water is sterilized by killing any bacteria that may be present. The outlet dispenser 24 is removed and a sterilizing manifold 48, as illustrated in FIGS. 2 and 5, is installed in its place between the outlet port 23 and sanitizing port 49 so that the heated water can be circulated throughout the entire system to sterilize the system. After the heated water has been circulated throughout the system for a predetermined period of time, the water within the system may be allowed to cool to either ambient room temperature or the desired water maintenance temperature, or the water may be discarded through drain 44, and new water brought into the water tank 14, which will be purified as described above using ultraviolet light and a series of filters. The water dispenser 24 can be sterilized, such as being autoclaved, before replacing the sterilizing manifold 48 for normal operation and use.

An electronic controller 50 is in electronic communication, or otherwise operably connected to, many of the components of the system so as to control the temperature of the water, the pump, and controllably dispense a desired amount of water from the unit 10, as desired. The term “electronic controller” as used herein may refer to a single unit, multiple units, integrated circuits, a computer processor, volatile and/or non-volatile memory, and the like commonly used in connection with electronic controllers and computers to monitor and control various components and subsystems and provide user interface and storage and retrieval of information. The electronic controller may comprise a programmable logic controller having memory associated therewith.

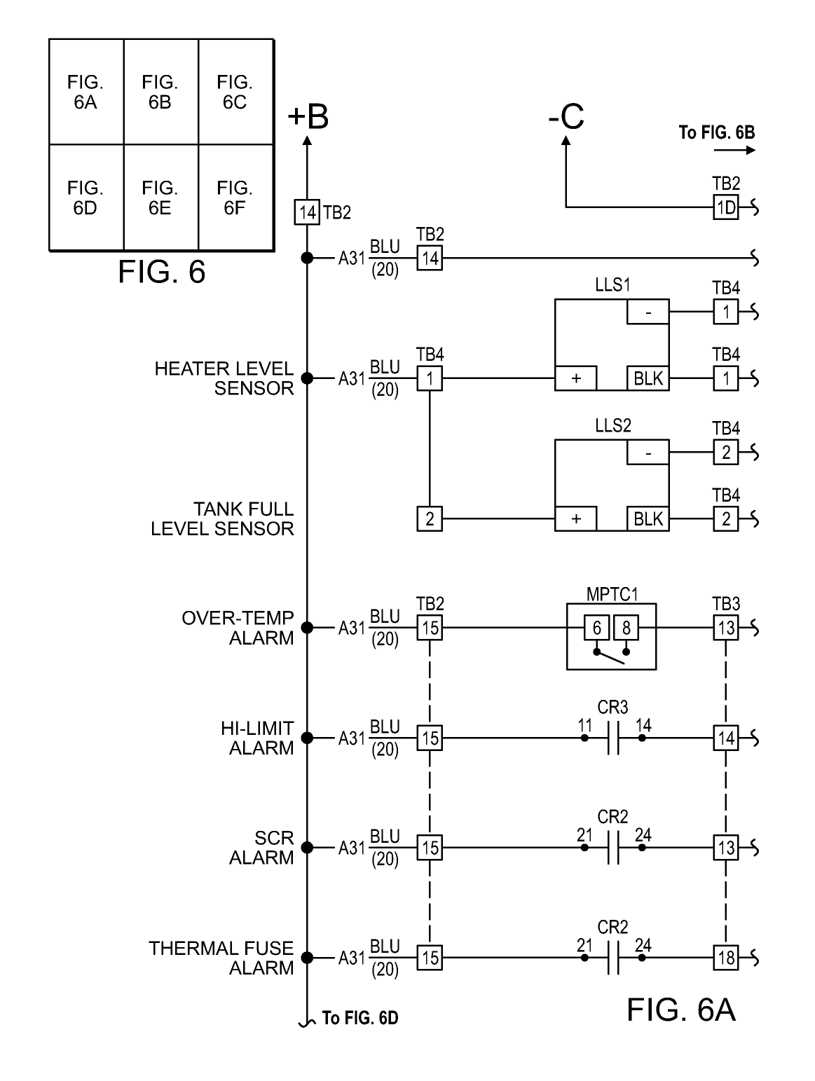

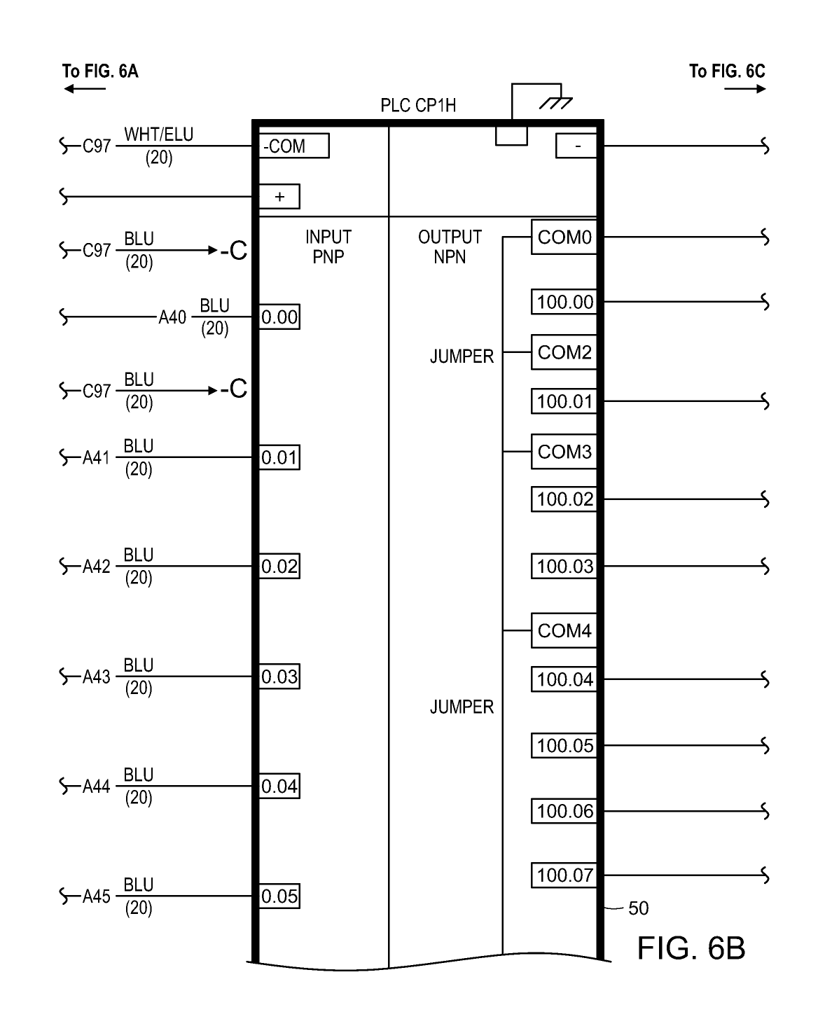

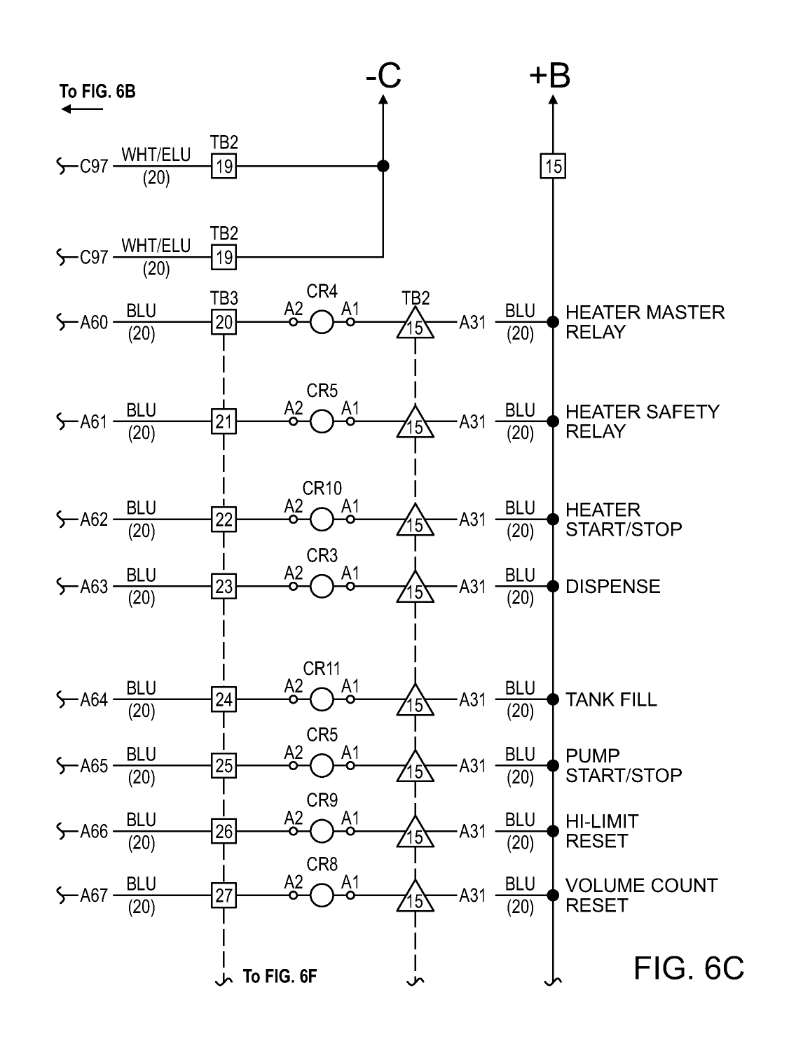

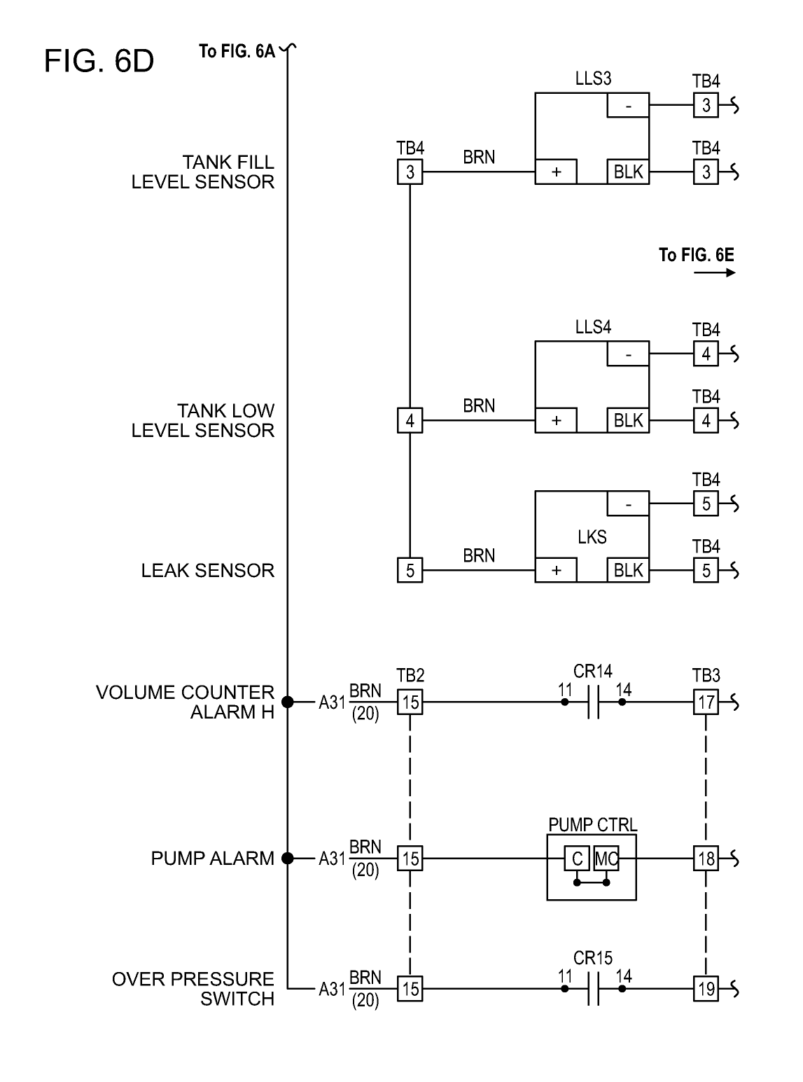

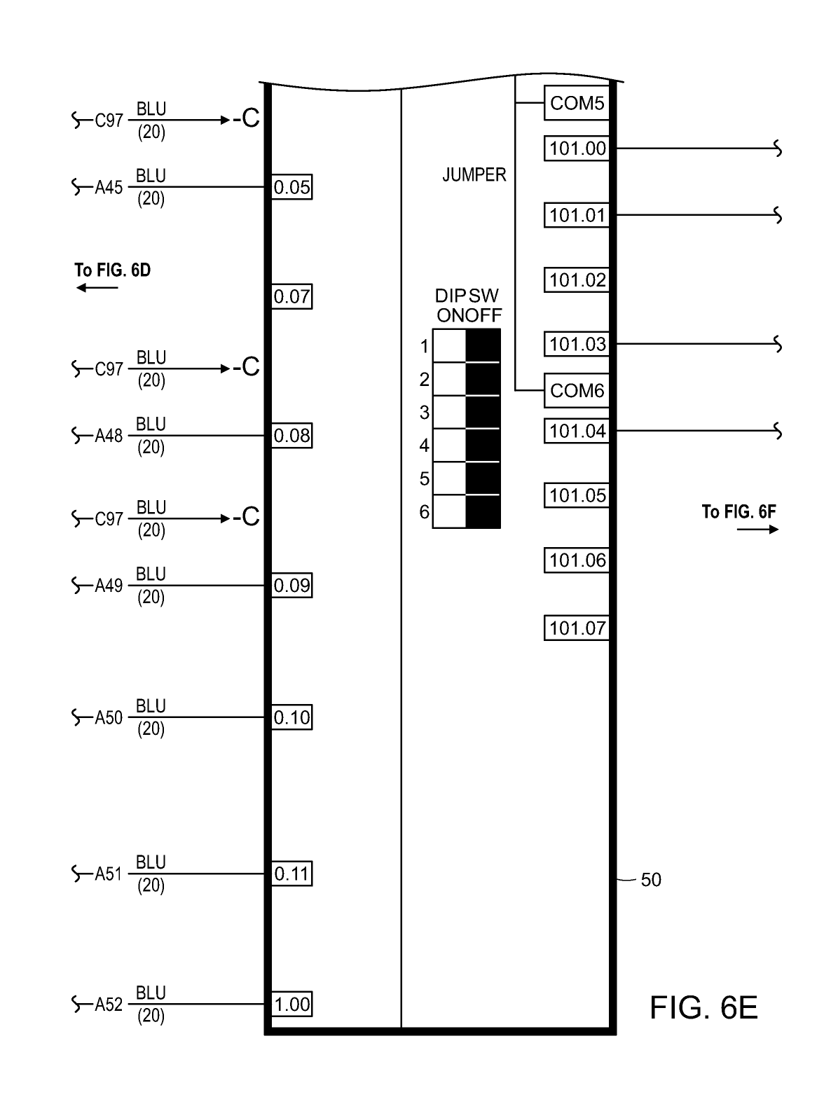

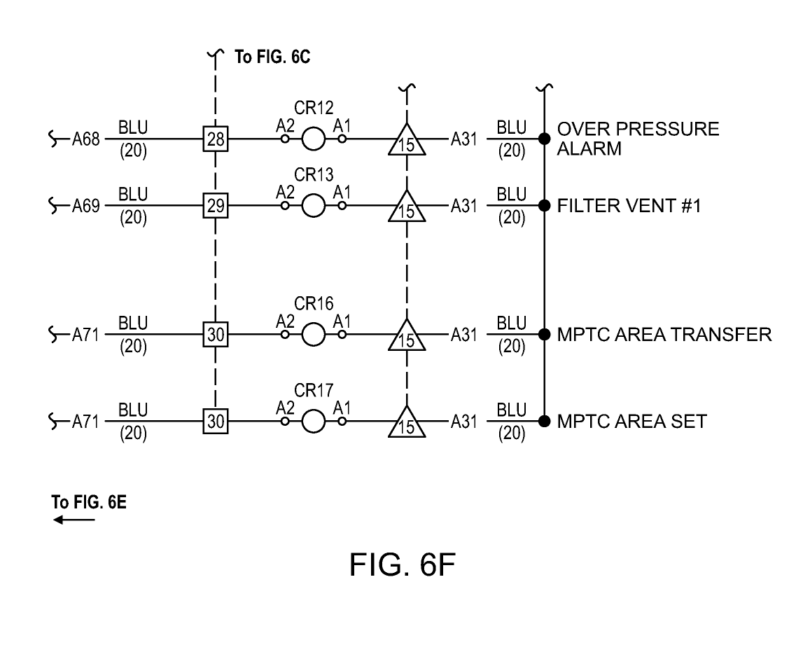

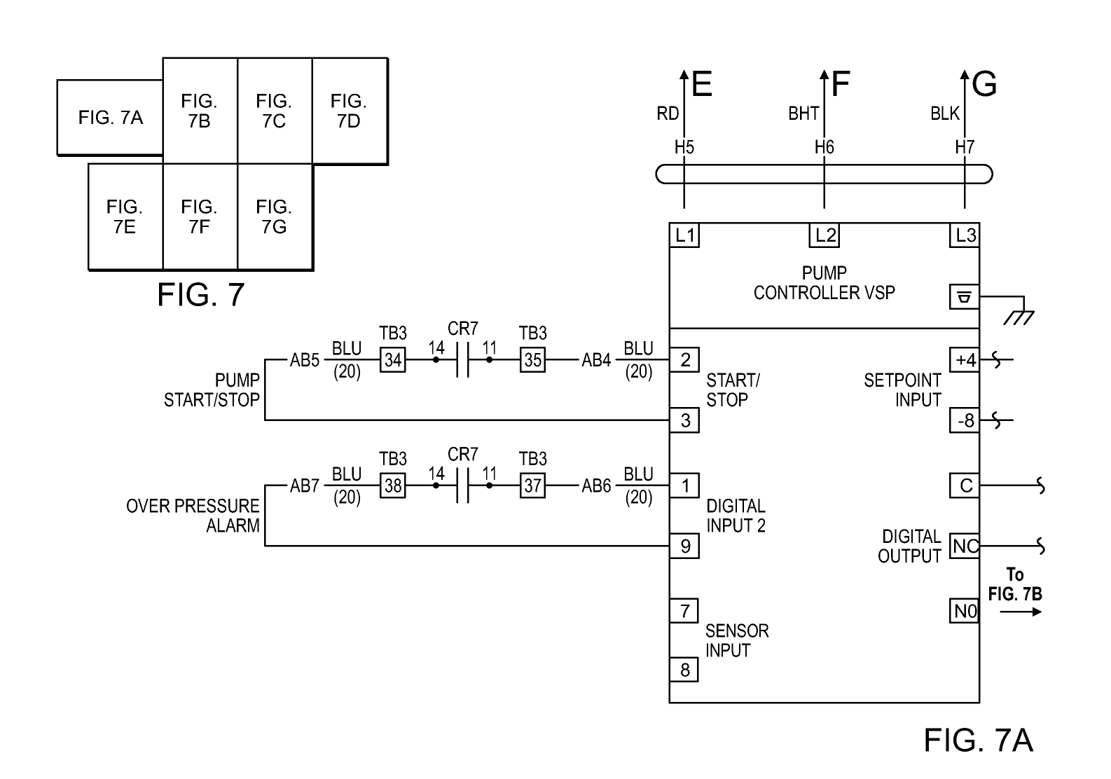

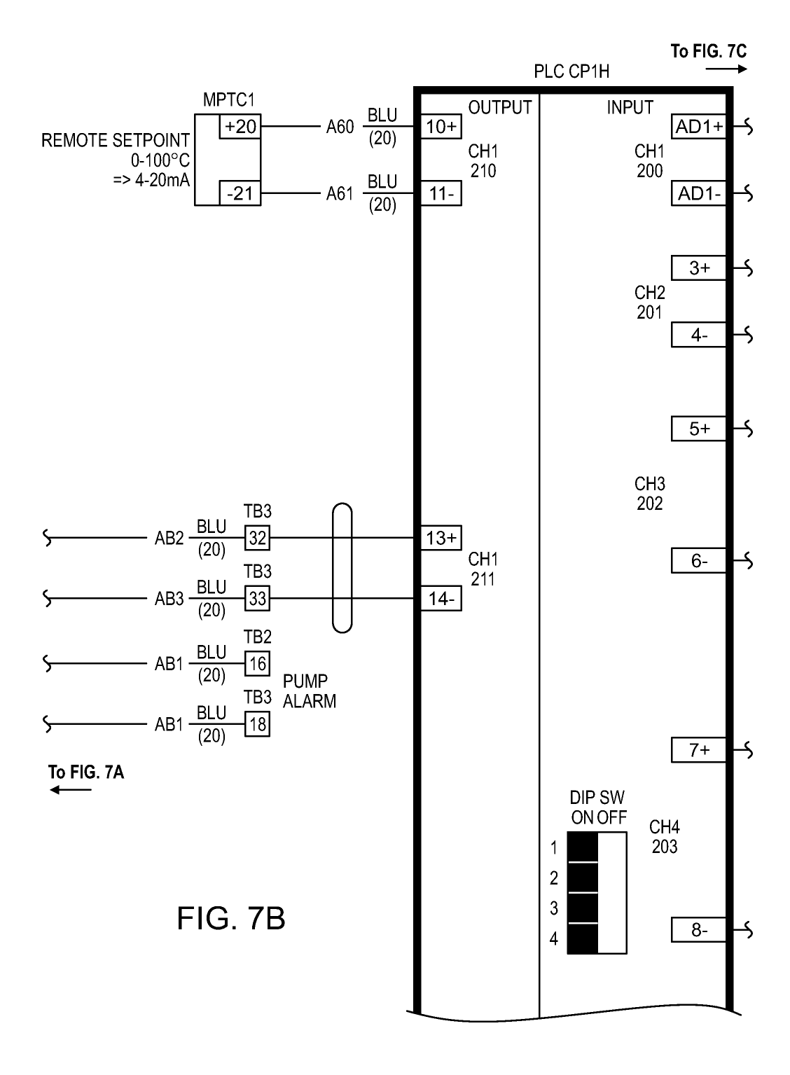

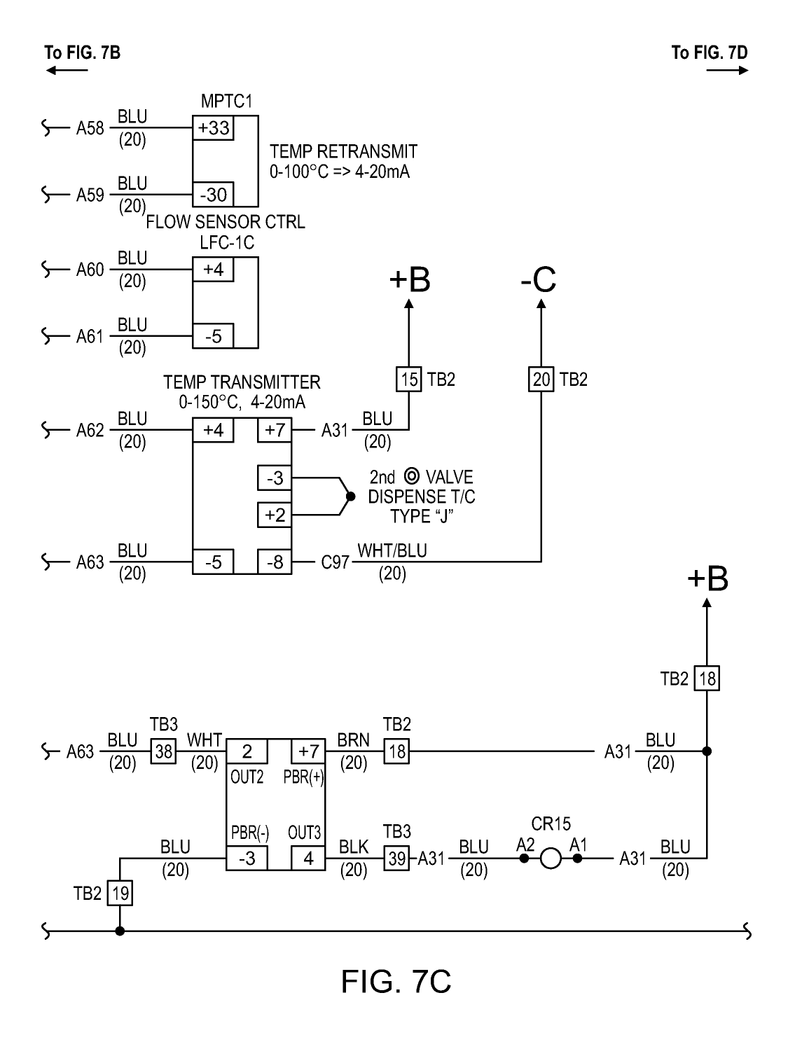

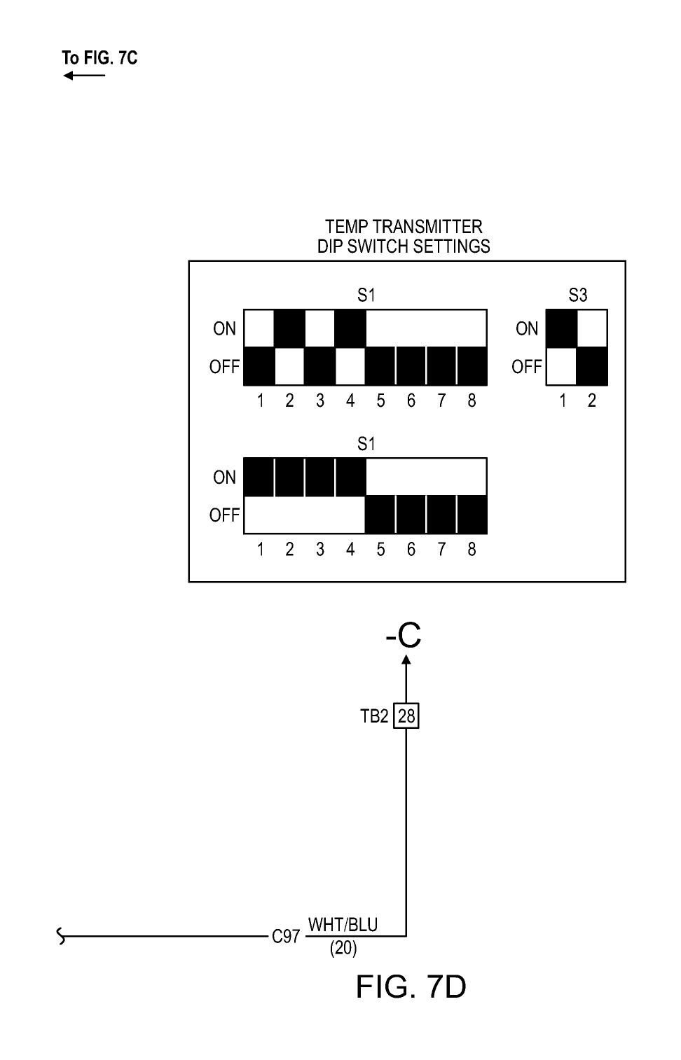

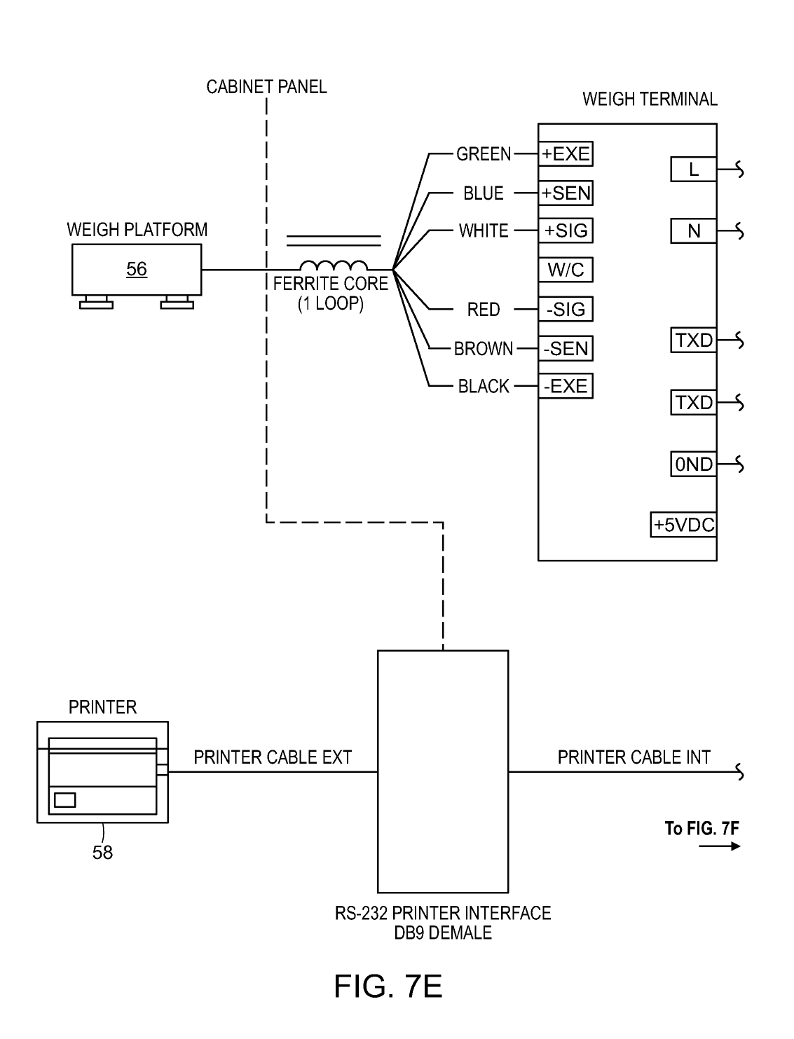

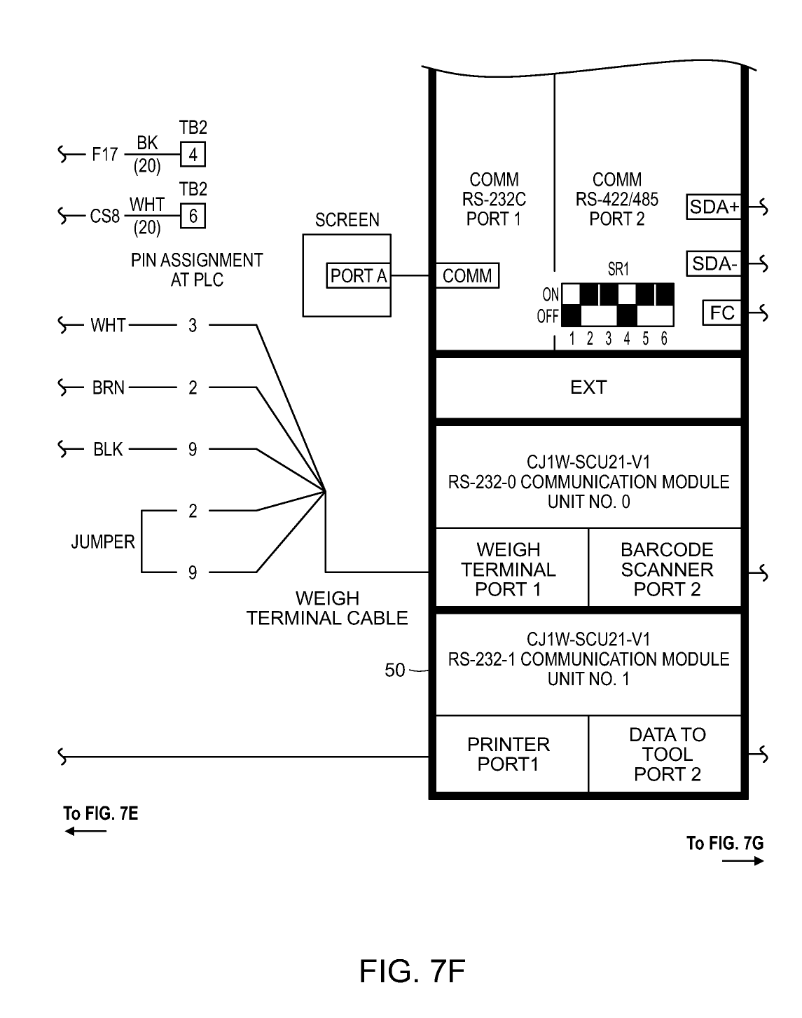

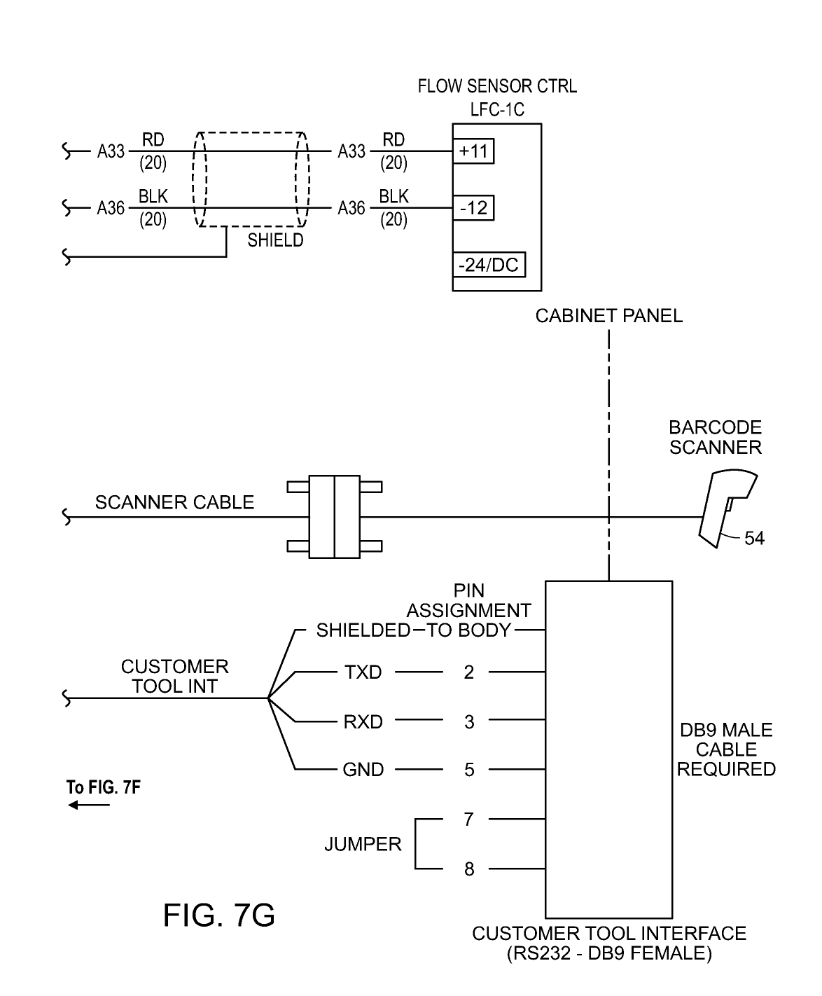

FIGS. 6A through 6F and 7A through 7G are schematics illustrating an exemplary electronic controller in electronic communication with various components and subsystems of the system of the present invention. The electronic controller, for example, controls the heating of the water to a desired temperature, using the heater, thermal couple assemblies, thermal fuses, and thermal sensors. A record is automatically maintained of the water dispensed, including the temperature at which the water is dispensed.

The electronic controller is also used to monitor the level of the water tank 14 and actuate the necessary valves and the like so as to introduce water into the water tank 14, as necessary, or to alert the operator of the need to fill the water tank 14 when its level is low. The electronic controller 50 may also be coupled to leak sensors or the like to detect any water leaks within the system.

The electronic controller 50 also serves to controllably dispense a selected and predetermined amount of water from the water dispenser 24. The electronic controller is in communication with a water flow sensor and a valve as well as the pump in order to dispense a very accurate amount of water.

Utilizing the electronic controller and sensors and valves, etc. of the present invention enables the operator to have an accuracy of the water dispensed from the system of plus or minus 2%, and a temperature accuracy of plus or minus 2° C. This greatly eliminates potential human error associated with manual processes for achieving an accurate amount of water added to the media as well as an accurate temperature of water being added to the media.

The system includes a laboratory information management system (LIMS) which is a software-based laboratory and information management system which works in conjunction with the electronic controller 50 in providing the automated processes of the system of the present invention. The computer program may be firmware and/or software. The LIMS serves to provide predetermined or previously saved “recipes” which correspond with different types of growth or enrichment culture media and microbes to be tested for. The amount of water to be dispensed and the temperature at which the water is to be dispensed can vary from one microbiological testing to another, depending upon the food or other item which is to be tested, and the microbe pathogens to be tested for, as well as the media which is to be used to incubate and to screen for the microbes being tested for.

The system includes one or more data entry devices in electronic communication with the electronic controller 50. The data entry device may comprise a keypad and an electronic display, or more typically a touchscreen 52 for the user to interface with the system and enter and/or select parameters, recipes and the like. In a particularly preferred embodiment, the system also includes a machine code reading scanner 54 which is capable of reading machine codes, such as barcodes, two-dimensional symbologies, QR codes, and the like. The scanner 54 may be used, for example, to scan a barcode or other machine-readable code associated with a media packet to be used in connection with a particular test. Upon scanning the machine readable code of the media, the information relating to the particular media and/or media packet will be conveyed to and stored at the electronic controller. The scanning of the machine-readable code associated with the packet of media may indicate a particular recipe to use in connection with the microbiology testing to be performed. In this case, the recipe of the volume of water to be dispensed and the temperature at which the water is to be dispensed may be automatically provided to the operator through the touchscreen 52 or other display for the operator to approve and select. Alternatively, the operator may input a desired volume of water to be dispensed and a desired temperature for the dispensed water.

There are benefits of using a quality certified premade media, such as media packets, as dehydrated media powder can be approximately one-half the price compared to premade media. Moreover, there is a time and cost savings by freeing up the autoclave process by using previously sterilized media. By not having to perform an autoclave step for each batch, time is saved and more samples may be processed in existing facilities in a given amount of time. Moreover, significant time savings may be attained from the incubator not having to bring the sample up to incubation temperature or being used to store pre-warmed media, as the water will be dispensed at a desired temperature which can correspond with the incubation temperature of the test. Preheating the water to approximately the incubation temperature can save up to several hours in incubation time.

As mentioned above, a very accurate level of water can be dispensed using the automated system of the present invention by utilizing the electronic controller 50 in communication and operation with solenoids, valves, flow sensors, pressure transducers, the pump speed and the like to dispense a very accurate amount of water, typically within 2% of the desired amount. The volume of water dispensed is saved in the memory of the system as part of the record established with respect to the test sample.

Moreover, a scale 56 is used to weigh the amount of water received within a container used for the test. The scale 56 typically provides several weight measurements, including the container and sample, the weight of the culture media added thereto, and finally the weight of the water dispensed therein. The scale 56 communicates to the electronic controller 50 the weight of the water dispensed into a container placed on the scale 56 to save as a record in the memory associated with the electronic controller 50.

Thus, both the volume of the water dispensed and the weight of the dispensed water is recorded as an extra verification step, in the event that the testing results are ever called into question. The record includes information electronically stored relating to the temperature of the water dispensed, the volume and weight of the dispensed water, the culture media to be mixed with the dispensed water, as well as the identity of the operator performing the test.

As an added level of precaution relating to the laboratory grade or purity of the dispensed water, the water is typically passed through a final filter 21 which filters objects larger than 0.2 microns shortly before the water is dispensed. This ensures that the water, which was previously purified by ultraviolet light as well as passing through at least one filter, is free from all bacteria which could disrupt the results of the microbiological test. While different types of media may be selected in order to target the growth of a particular microbe to be tested for, the presence of other bacteria could also be grown in the media which could create false positive readings or require that the test be repeated. This is avoided by dispensing laboratory-grade, purified water in which all objects, including bacteria, having a size greater than 0.2 microns are filtered from the system. In a particularly preferred embodiment, as described above, objects having a greater size than 0.1 microns are filtered from the system. Moreover, exposure to the ultraviolet light will kill or disable all microbes. Thus, the system of the present invention dispenses very pure water to ensure that the test results are accurate.

In accordance with the present invention, an operator logs into the system, such as by scanning a barcode or other machine-readable code associated with a badge, such as by scanning the code using the scanner 54, or entering in a name, identification code or the like into the touchscreen 52. A testing recipe is selected or entered into the system. A time and date will be read from an internal system clock and recorded into the memory storage space associated with the record as well. Time and date storage occurs automatically without the need of the operator to input this information.

A sample, such as a sample of food is added to a container disposed on the scale 56. The scale 56 takes a measurement of the weight of the sample. Media, such as from a media packet which has been premeasured and sterile, is then added to the container. Information relating to the media powder added to the sample is recorded, such as by scanning the machine-readable code associated with the media packet. The scale 56 weighs the weight of the media added, and relays this to the electronic controller and to the record of the test.

Purified and sterilized water is then dispensed at the desired volume and temperature. The water from the water tank 14 feeds a pump 22, and the water exiting the pump comes into contact with a pressure relief valve and a pressure transducer, then flows into the in-line heater 46 followed by the final filter which is rated at 0.2 microns. The water then leads through a flow sensor and a three-way air operated valve. If there is no demand, the water will flow back to the water tank 14 and circulate again through the system. However, if there is demand and the water is to be dispensed, the water will flow through the dispense port or spout 24. “Demand” means that a food sample is ready for testing and that the media preparation must take place.

Selecting a previously stored recipe may indicate the water volume and water temperature. Alternatively, the operator may select the water volume and water temperature on the touchscreen before dispensing. The system is such that the circulating water is controlled by the speed of the pump. Once the user selects the dispense volume, the controller adjusts the pump speed to optimize the dispense and resolution of the flow sensor and the totalizer in the flow sensor. With the speed of the pump set, the air operated valve is open to allow dispense to occur. The dispense will continue until the flow sensor totalizer detects that the desired volume has been provided, at which point a bit is sent to the controller to close the air operated valve. Once the correct amount of laboratory-grade water is dispensed, the scale weighs the container again to provide a weight measurement of the dispensed water.

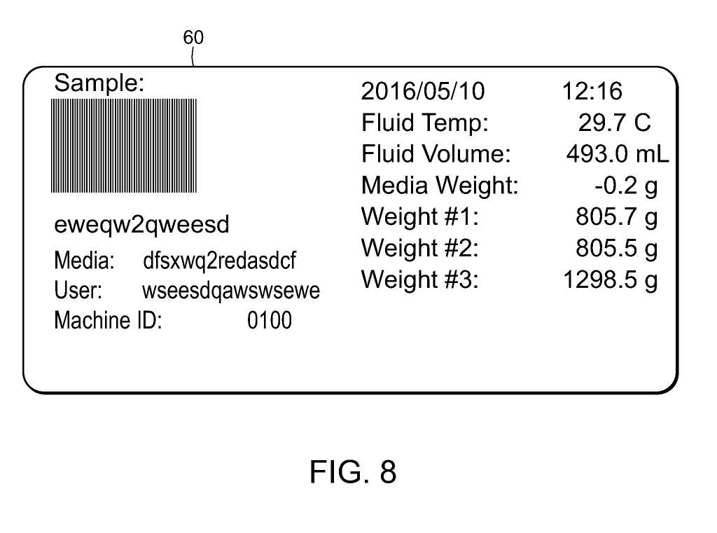

A printer 58 may be in electronic communication with the electronic controller for the printing of a label 60 or the like, such as that illustrated in FIG. 8. Typically, for each sample tested, a label 60 is printed which includes information relating to the record, including user or operator identification, culture media identification, machine identification, date, time, water temperature, water volume, container/sample weight, container/sample/sample media weight, and weight of the container/sample/media/water. One or more labels can be affixed to the user's log books for traceability and/or to the container. If the results of the testing are ever called into question, the information relating to the sample test can be obtained from the one or more labels associated with the test, either on the container, operator's log books, or even through a soft copy record maintained in the LIMS associated with the electronic controller. Logging the media preparation test procedural data provides quicker and higher accuracy recordkeeping compared to handwritten laboratory notebooks.

Although several embodiments have been described in detail for purposes of illustration, various modifications may be made without departing from the scope and spirit of the invention. Accordingly, the invention is not to be limited, except as by the appended claims.

What is claimed is:

1. An automated culture media preparation system for microbiology testing, comprising:
a water tank, including a water inlet, a water outlet and an air vent;
an air filter associated with the air vent for filtering bacteria from the air entering the water tank;
a water dispenser in fluid communication with the water tank;
a pump for circulating water and moving the water from the water tank to the dispenser;
means for filtering, killing and/or disabling bacteria within the water;
an electronic controller;
a heater in electronic communication with the electronic controller for heating the water to a selected temperature; and
means for controllably dispensing a selected amount of water from the water dispenser, the dispensing means being operably coupled to the electronic controller.

2. The system of claim 1, including a scale associated with the water dispenser.

3. The system of claim 2, wherein the scale communicates with the electronic controller so as to record the weight of the water dispensed into a container placed on the scale in memory associated with the electronic controller.

4. The system of claim 1, including a printer for printing labels, the printer being in electronic communication with the electronic controller.

5. The system of claim 1, including a data entry device comprising a keypad, a touchscreen, and/or a machine code reading scanner in electronic communication with the electronic controller.

6. The system of claim 1, including a water temperature sensor operably coupled to electronic controller.

7. The system of claim 1, wherein the controllably dispensing means comprises a flow sensor and a valve operably coupled to the electronic controller.

8. The system of claim 1, wherein the electronic controller comprises a programmable logic controller having memory associated therewith.

9. The system of claim 1, wherein the filtering, killing and/or disabling means comprise a filter capable of filtering bacteria from the water and/or a source of energy that kills or disables the bacteria.

10. The system of claim 9, wherein the filtering, killing and/or disabling means comprise a filter capable of filtering bacteria from the water and a source of ultraviolet energy that kills or disables the bacteria disposed in series between the water tank inlet and the water dispenser.

11. The system of claim 9, wherein the filtering, killing and/or disabling means comprise a first filter that filters the water of objects having a size of greater than 1 micron and a second filter that filters the water of objects having a size greater than 0.2 microns.

12. The system of claim 1, wherein the air filter associated with the air vent comprises an ultra-low penetration filter capable of filtering objects having a size greater than 0.2 microns.

13. A process for preparing media for microbiology testing, comprising the steps of:
providing a source of water;
purifying the water of pathogenic bacteria;
heating the water to a predetermined temperature;
dispensing a predetermined amount of the purified and heated water into a container for mixing with a culture media;
weighing the water dispensed into the container;
electronically storing information relating to the temperature, volume and weight of the dispensed water and the culture media to be mixed with the dispensed water; and
printing a label having indicia, including the electronically stored information.

14. The process of claim 13, wherein the providing water step comprises the steps of providing a water tank exposed to air filtered for objects at least the size of a bacteria.

15. The process of claim 13, wherein the purifying step comprises the step of filtering, disabling and/or killing bacteria within the water.

16. The process of claim 15, wherein the purifying step comprises the step of passing the water through a filter that filters bacteria from the water.

17. The process of claim 16, including the step of passing the water through a series of filters so that objects that are at least 0.2 microns in size are filtered from the water.

18. The process of claim 13, wherein the purifying step comprises the step of exposing the water to ultraviolet light to kill or disable bacteria within the water.

19. The process of claim 13, wherein the water is heated to a temperature corresponding with an incubation temperature of bacteria to be tested for.

20. The process of claim 13, including the step of preventing water stagnation by circulating the water through a device that stores and dispenses the water.

21. The process of claim 13, including the step of sterilizing a device that stores and dispenses the water by heating the water to a temperature sufficient to kill or disable bacteria and circulating the heated water through the device.
